(19) United States
(12) Patent Application Publication
(10) Pub. No.: US 2019/0209595 A1
(43) Pub. Date: Jul. 11, 2019

(54) ORALLY ADMINISTRABLE FORMULATIONS FOR THE CONTROLLED RELEASE OF A PHARMACOLOGICALLY ACTIVE AGENT

(71) Applicant: Vivus, Inc., Campbell, CA (US)

(72) Inventors: Calvin Theodore BROMAN, Saratoga, CA (US); Eric SHEU, Campbell, CA (US)

(21) Appl. No.: 16/108,396

(22) Filed: Aug. 22, 2018

Related U.S. Application Data
(63) Continuation of application No. 14/713,938, filed on May 15, 2015, now abandoned.
(60) Provisional application No. 61/994,347, filed on May 16, 2014.

(51) Int. Cl. A61K 31/7048; A61K 31/137; A61K 9/24; A61K 9/20; A61K 45/06; B29C 43/00

(52) U.S. Cl. CPC A61K 31/7048; A61K 31/137; A61K 9/209; A61K 9/2013; B29L 2031/753; A61K 45/06; B29C 43/006; A61K 9/2018; A61K 9/2095; A61K 9/2009

(57) ABSTRACT

Drug tablets that include a controlled release layer of a moisture-sensitive active agent are prepared with a lipidic matrix forming excipient, a water-soluble, channel forming excipient and a filler, each being non-hygroscipic. The tablets are formed in a process where the components are blended in the absence of moisture and in particulate form.

### CROSS-REFERENCE TO RELATED APPLICATIONS

This application is a continuation of U.S. application Ser. No. 14/713,938 filed May 15, 2015, which claims priority to, and the benefit of, U.S. Provisional Patent Application Ser. No. 61/994,347 filed May 16, 2014, the entire contents of each of which are herein incorporated by reference in their entireties.

### FIELD OF INVENTION

The disclosure relates to, among other things, pharmaceutical compositions, and more particularly relates to controlled release formulations and drug delivery systems for the oral administration of pharmacologically active agents.

### BACKGROUND OF THE INVENTION

The prevalence of obesity in children and adults is on the rise in many nations, including both developed nations such as the United States and developing nations such as China and India. Obesity can be associated with a variety of medical problems, such as one or more of diabetes, shortness of breath, asthma, pulmonary hypertension, gallbladder disease, dyslipidemia, for example, hyperchloesteremia, dyslipidic hypertension, osteoarthritis, reflux esophagitis, snoring, sleep apnea, menstrual irregularities, infertility, pregnancy complications, gout, coronary artery disease, heart disease, muscular dystrophy, metabolic disorders such as hypoalphalipoproteinemia, familial combined hyperlipidemia, and Syndrome X, for example, insulin-resistant Syndrome X. Obesity is also associated with increased incidence of some cancers, such as cancers of the colon, rectum, prostate, breast, uterus, and cervix. Obese subjects can also suffer emotional problems related to societal reactions towards obesity.

In addition to being related to incidence of various diseases, obesity can increase the risk of death from hypertension, dyslipidemia, diabetes, such as type II diabetes mellitus, coronary artery disease, heart disease, stroke, gallbladder disease, osteoarthritis, liver disease, and cancers, such as endometrial, breast, prostate, and colon cancers (see, for example, Pi-Sunyer et al. Postgrad Med 2009:121:21-33). Obesity can also be associated with increased all-cause mortality.

Topiramate, a sulfamate-substituted monosaccharide with the chemical name 2,3,4,5-bis-O-(1methyletylidene)-β-D-fructopyranose sulfamate, has been reported for use in treating obesity and promoting weight loss, for example, in U.S. Pat. Nos. 7,056,890, 8,580,298, and 8,580,299, and is also marketed for treating migraine headaches and seizure related disorders. A variety of dosages of topiramate can be used for these purposes, depending on the weight, age, gender, and other characteristics of the subject. Although efficacious for these purposes, topiramate is known to have harmful side effects in some subjects. Furthermore, some subjects do not respond to topiramate treatment for obesity. Thus, there is a need for a dosing regimen for topiramate that minimizes subjects' exposure to topiramate while providing one or more indications of whether a particular subject is likely to experience harmful side effects and/or respond to topiramate treatment. The embodiments described herein can meet these and other needs. Topiramate, 2,3:4,5-bis-O-(1-methylethylidene)-beta-D-fructopyranose sulfamate, was originally described in U.S. Pat. No. 4,513,006, along with its use in treating epilepsy and glaucoma. Topiramate, as Topamax® (Johnson & Johnson Corp.), has been approved by the US FDA as a migraine medication as well as a treatment for epilepsy and glaucoma. Topiramate has also been proposed for use in treating other conditions, such as bipolar disorder, neuropathic pain, impulse control disorders, psoriasis, and amyotrophic lateral sclerosis. See U.S. Pat. No. 6,699,840 to Almarsson et al.; U.S. Pat. No. 6,323,236 to McElroy et al.; U.S. Pat. No. 5,760,006 to Shank et al.; and U.S. Pat. No. 5,753,694 to Shank et al.

Recently, a new formulation of topiramate, in combination with a second agent, phentermine, has been developed and is now commercially available as a medication for the treatment of obesity and potentially related conditions such as type 2 diabetes (QSYMIA®, available from Vivus, Inc., Mountain View, Calif.). Qsymia is currently available as a capsule containing controlled release topiramate beads and phentermine in immediate release form. See U.S. Pat. Nos. 7,056,890, 7,553,818, 7,659,256, and 7,674,776 to Najarian and U.S. Pat. Nos. 8,580,298, and 8,580,299 to Narjarian et al.

It is well known that topiramate is chemically and physically stable in the solid state in ambient conditions, but tends to degrade in the presence of moisture, presumably as a result of an impurity in the synthesized product. See Micheel et al. (1998) J. Chromatogr. B Biomed. Sci. Appl. 709 (1):166-172. Consequently, there is an ongoing need for topiramate formulations that have enhanced stability in the presence of moisture, particularly controlled release topiramate formulations that provide for gradual release of the active agent over an extended time period.

U.S. Pat. No. 7,125,560 to Thakur et al. describes taste-masked, chewable topiramate formulations, and notes that one of the reasons a chewable form of topiramate had been difficult to formulate is the instability of topiramate upon exposure to moisture and heat. The patent describes preparing topiramate cores via roller compaction and comminution or, alternatively, via extrusion-spheronization, followed by application of a taste-masking coating, stated to enhance the stability of the topiramate core. The taste-masked topiramate particles are described as usable in a “sprinkle formulation” to be dispersed onto food, and as compressible to provide chewable tablets, both of which provide for immediate release of the active agent.

US Patent Publication No. 2007/0243254A1 to Edgren et al. describes a controlled release topiramate composition in the form of an osmotic drug delivery system. In such a system, as water flows through a semipermeable membrane into an encapsulated osmotic core containing the active agent, hydrostatic pressure builds within the core, and a saturated solution of the agent is continuously released through a drug delivery orifice. US Patent Publication No. 2008/0085306A1 to Nangia et al. also describes a controlled release topiramate composition in the form of an osmotically controlled drug delivery system.

Osmotic drug delivery systems have numerous drawbacks, however, including a limited selection of excipients and semipermeable polymer membranes, as well as a low limit on drug loading, generally equivalent to at most about 25% of the tablet by weight, substantially less than is possible with a matrix tablet. See Shamblin, “Controlled Release Using Bilayer Osmotic Tablet Technology: Reducing Theory to Practice,” in Oral Controlled Release Formulation Design and Drug Delivery: Theory to Practice, Eds. H. Wen and K. Park (Hoboken, N.J.: John Wiley & Sons, Inc., 2010).

An ideal controlled release topiramate formulation would exhibit superior stability with respect to moisture, be straightforward and economical to manufacture, and not limited by the constraints inherent in osmotic delivery systems. Such a formulation would also be useful for delivering other moisture-sensitive active agents as well.

For a combination formulation containing topiramate and a second agent, e.g., phentermine, as in QSYMIA®, where one agent is most effectively administered according to an immediate release profile (i.e., phentermine, a stimulant), and the second agent is most effectively administered according to a sustained release profile (i.e., topiramate), it would be ideal if the two profiles could be achieved with a single dosage form in which substantially the same excipients and method of manufacture could be used with each active agent. That is, an ideal dosage form would contain topiramate in controlled release form and phentermine in immediate release form, with each agent present in a discrete region of the dosage form containing substantially the same excipients and manufacturable using substantially the same method.

### SUMMARY OF THE INVENTION

In some embodiments, an orally administrable compressed tablet for controlled release of a moisture-sensitive pharmacologically active agent is disclosed. The table may include a non-hygroscopic, lipidic matrix-forming excipient having a melting point greater than about 40° C., a non-hygroscopic, water-soluble, channel-forming excipient, a non-hygroscopic filler, and a therapeutically effective amount of the pharmacologically active agent, wherein the tablet is substantially free of hygroscopic excipients.

In an additional embodiment, a particulate formulation for forming a compressed tablet is disclosed. The formulation has, in particulate form, a non-hygroscopic, lipidic matrix-forming excipient having a melting point greater than about 40° C., a non-hygroscopic, water-soluble, channel-forming excipient, a non-hygroscopic filler; and a therapeutically effective amount of a moisture-sensitive pharmacologically active agent, wherein the formulation is substantially free of hygroscopic excipients.

In another aspect, an orally administrable compressed tablet for controlled release of topiramate is disclosed. The tablet may include, comprising a non-hygroscopic, lipidic matrix-forming excipient having a melting point greater than about 40° C., selected from glyceryl mono-esters, di-esters, and tri-esters of C₁₂-C₂₆ fatty acids, a non-hygroscopic, water-soluble, channel-forming excipient selected from mannitol, sorbitol, galactitol, fucitol, iditol, inositol, volemitol, alpha-D-glucopyranosido-1,6-sorbitol, alpha-D-glucopyranosido-1,6-mannitol, isomalt, and combinations thereof, a non-hygroscopic filler; and a therapeutically effective amount of topiramate, wherein the tablet is substantially free of hygroscopic excipients.

In an additional embodiment, a process for manufacturing an orally administrable compressed tablet for controlled release of a moisture-sensitive pharmacologically active agent is disclosed. The method comprises (i) blending, in the absence of moisture and in particulate form, a non-hygroscopic, lipidic matrix-forming excipient having a melting point greater than about 40° C., with a non-hygroscopic, water-soluble, channel-forming excipient, a non-hygroscopic filler, and the moisture-sensitive active agent, to form an active agent mixture, (ii) incorporating at least one manufacturing aid into the active agent mixture to provide a controlled release formulation; and (iii) subjecting the formulation to compressive forces to give a controlled release dosage form for administration of the moisture-sensitive active agent.

In a particular embodiment, a process for manufacturing an orally administrable compressed tablet for controlled release of a moisture-sensitive pharmacologically active agent and immediate release of a second pharmacologically active agent is disclosed. The process includes that steps of (i) blending, in the absence of moisture and in particulate form, a non-hygroscopic, water-soluble, channel-forming excipient, a non-hygroscopic filler, and manufacturing aids, to form an initial excipient mixture; (ii) dividing the initial excipient mixture into a first batch and a second batch; (iii) incorporating a lipidic matrix-forming excipient and a moisture-sensitive active agent into the initial excipient mixture, to form a controlled release formulation; (iv) incorporating a second active agent into the second batch, to form an immediate release formulation; (v) applying compressive force to a contained quantity of the controlled release formulation to form a controlled release layer; (vi) applying compressive force to a contained quantity of the immediate release formulation to form an immediate release layer; and (vii) compressing the immediate release layer to the controlled release layer in a manner that facilitates bonding of the two layers.

In a particular embodiment, a bilayer tablet having 23 mg of topiramate is formulated with a sustained release topiramate layer and an immediate release layer of phentermine hydrochloride using the following formulation: (22.6%) calcium sulfate dehydrate, (16%) glyceryl behenate, (24.1% silicified microcrystalline cellulose, (16%) isomalt, (0.8%) magnesium stearate, (0.5%), fumed silica and 20% topiramate. This tablet (formulation A/mTPM-001d) has a dissolution profile similar to the currently commercialized capsule.

In another embodiment a bilayer tablet having 23 mg of topiramate is formulated with a sustained release topiramate layer and an immediate release layer of phentermine hydrochloride using the following formulation: calcium sulfate dehydrate (22.6%), glyceryl behenate (20.0%), silicified microcrystalline cellulose (24.1%), isomalt (12.0%), magnesium stearate (0.8%), fumed silica (0.5%), and topiramate (20%). This formulation, formulation B/mTPM-001e has a dissolution profile that is slower than the currently commercialized capsules).

In another embodiment, High dose Formulation (92 mg) C: Calcium sulfate dehydrate (21.43%), Glyceryl behenate (5.19%), silicified microcrystalline cellulose (15%), isomalt (24.59%), magnesium stearate (0.68%), fumed silica (0.43%), Topiramate (32.69%) (Similar to dissolution profile of capsules). (formulation C)

In another embodiment a bilayer tablet having 92 mg topiramate is formulated with a sustained release topiramate layer (formulation D/mTPM-002f): Calcium sulfate dehydrate (18.26%) Glyceryl behenate (11.97%), silicified microcrystalline cellulose (13%), isomalt (25.47%), magnesium stearate (0.80%), fumed silica (0.5%), Topiramate (30.0%) (dissolution profile slower than in capsules).

### BRIEF DESCRIPTION OF THE DRAWINGS

FIG. 1 is a schematic of an example of a tablet from a side view showing exemplary dimensions.

FIG. 2 shows percent dissolution results for tablets having formulations as provided in Examples 1-7.

### DETAILED DESCRIPTION

In this application, including the appended claims, the singular forms “a,” “an,” and “the” are often used for convenience. However, it should be understood that these singular forms include the plural unless otherwise specified. It should also be understood that all patents, publications, journal articles, technical documents, and the like, referred to in this application, are hereby incorporated by reference in their entirety and for all purposes.

Unless otherwise defined, all terms used in this application should be given their standard and typical meanings in the art, and are used as those terms would be used by a person of ordinary skill in the art at the time of the invention.

“Active agent” as used herein encompass not only the specified molecular entity but also its pharmaceutically acceptable, pharmacologically active analogs, including, but not limited to, salts, esters, amides, prodrugs, conjugates, active metabolites, and other such derivatives, analogs, and related compounds as will be discussed infra. Therefore, reference to “phentermine,” for example, encompasses not only phentermine per se but also salts and other derivatives of phentermine, e.g., phentermine hydrochloride. It is to be understood that when amounts or doses are specified, that those amounts or doses refer to the amount or dose of active agent per se and not to a salt or the like. For example, when it is indicated that a dose or amount of phentermine is 7.5 mg, that would correspond to 9.33 phentermine hydrochloride and not 7.5 phentermine hydrochloride. The molecular weight of phentermine is 149.23 g/mol and the molecular weight of phentermine hydrochloride is 185.69 g/mol.

“Administering” as used herein includes to any route of administration, for example, oral, parenteral, intramuscular, transdermal, intravenous, inter-arterial, nasal, vaginal, sublingual, subungual, etc. Administering can also include prescribing a drug to be delivered to a subject, for example, according to a particular dosing regimen, or filling a prescription for a drug that was prescribed to be delivered to a subject, for example, according to a particular dosing regimen.

“Body Mass Index” or “BMI” as used herein is an index of weight-for-height that is commonly used to classify overweight and obesity in adults. BMI may be calculated by multiplying an individual's weight, in kilograms, by height, in meters. Currently the CDC and WHO define obesity as having a BMI of 30 or higher. A BMI between 25 and 29.9 is considered overweight. A BMI over 40 is sometimes characterized as morbidly obese. Individuals having a BMI between 30 and 35 may also be referred to as moderately obese, from 35 to 40 severely obese and over 40 very severely obese.

A “daily dose” of a particular material refers the amount of the material administered in a day. A daily dose can be administered as a single dose or as multiple doses. When a daily dose is administered as multiple doses, the daily dose is the sum of the amount of material administered in all of the multiple doses that are administered over the course of one day. For example, a daily dose of 12 mg can be administered in a single 12 mg dose once per day, in 6 mg doses administered twice per day, in 4 mg doses administered three times per day, in 2 mg doses administered six times per day, etc. The multiple doses can be the same or different doses of the material, unless otherwise specified. When a daily dose is administered as multiple doses, the multiple doses can be administered by the same or different route of administration, unless otherwise specified. Thus, a daily dose of 12 mg can include, for example, a 10 mg intramuscular dose and a 2 mg oral dose administered over the course of one day.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release, modified release and delayed release formulations.

Administration of one compound “with” a second compound, as used herein, includes but is not limited to cases where the two compounds are administered simultaneously or substantially simultaneously. For example, administration of a first compound with a second compound can include administering the first compound in the morning and administering the second compound in the evening, as well as administering the first and second compounds in the same dosage form or in two different dosage forms that at the same or nearly the same time.

“Topiramate” as used herein includes not only the chemical compound 2,3,4,5-bis-O-(1methyletylidene)-β-D-fructopyranose sulfamate, but also all stereoisomers, such as enantiomers and diastereomers, thereof, as well as salts, mixed salts, polymorphs, solvates, including mixed hydrates and mixed solvates, of one or more stereoisomers or mixtures of stereoisomers.

“Phentermine” as used herein includes not only the chemical compound 2-methyl-1-phenylpropan-2-amine, but also all stereoisomers, such as enantiomers and diastereomers, thereof, as well as salts, mixed salts, polymorphs, solvates, including mixed hydrates and mixed solvates, of one or more stereoisomers or mixtures of stereoisomers.

A “subject” or multiple “subjects” can be members of any species, typically human. The subjects of all experiments and studies discussed herein were human except when otherwise indicated.

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period. The term “delayed release” is also used in its conventional sense, to refer to a drug formulation which, following administration to a patient provides a measurable time delay before drug is released from the formulation into the patient's body.

The term “excipient” refers to a compound or composition in a pharmaceutical formulation or dosage form that is not intended to have biological activity itself.

The term “non-hygroscopic” means the ability of a solid or semi-solid compound or composition to withstand exposure to the water vapor of an ambient atmosphere for 24 hours or longer without giving rise to adverse phenomena such as aggregation, agglomeration, water absorption, or deliquescence. An excipient can be confirmed as non-hygroscopic using the foregoing test, i.e., exposure to water vapor for 24 hours, or using other means, e.g., dynamic vapor sorption (DVS). It will be appreciated by those skilled in the art that a non-hygroscopic material exhibits a DVS isotherm in which the water content of the material changes by less than 5% over a relative humidity (RH) range of 0% to about 75%, preferably over an RH range of 0% to about 60%. A non-hygroscopic material may absorb some moisture.

An excipient can be confirmed as non-hygroscopic using the foregoing test, i.e., exposure to water vapor for 24 hours, or using other means, e.g., dynamic vapor sorption (DVS). It will be appreciated by those skilled in the art that a non-hygroscopic material exhibits a DVS isotherm in which the water content of the material changes by less than 5% over a relative humidity (RH) range of 0% to about 75%, preferably over an RH range of 0% to about 60%.

The terms “hydrophilic” and “hydrophobic” are generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water, while hydrophobic compounds will generally have a P greater than about 1.0, typically greater than about 5.0. The term “lipidic” is used interchangeably with the term “hydrophobic” herein.

The terms “treating” and “treatment” include the following actions: (i) preventing a particular disease or disorder from occurring in a subject who may be predisposed to the disease or disorder but has not yet been diagnosed as having it; (ii) inhibiting the disease, i.e., arresting its development; or (iii) relieving the disease by reducing or eliminating symptoms and/or by causing regression of the disease.

The terms “effective amount” and “therapeutically effective amount” of a pharmacologically active agent refers to an amount that is nontoxic and effective for producing a therapeutic effect upon administration to a subject.

The term “dosage form” denotes any form of a pharmaceutical composition that contains an amount of active agent sufficient to achieve a therapeutic effect with a single administration of one, two, or more dosage forms. The frequency of administration that will provide the most effective results in an efficient manner without overdosing will vary with the characteristics of the particular active agent, including both its pharmacological characteristics and its physical characteristics, such as hydrophilicity. When the formulation is a tablet or capsule, the dosage form is usually one such tablet or capsule, although this is not required unless otherwise specified.

The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of topiramate, such that the unit dosage in each tablet or capsule is approximately 50% of the therapeutically effective amount.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations. That is, “controlled release” includes “sustained release” (synonymous with “extended release”), referring to a formulation that provides for gradual release of an active agent over an extended period of time, as well as “delayed release,” indicating a formulation that, following administration to a patient, provides for a measurable time delay before the active agent is released from the formulation into the body of the patient, e.g., the eye.

By “pharmaceutically acceptable” is meant a material that is not biologically or otherwise undesirable, i.e., the material may be incorporated into a pharmaceutical formulation administered to a patient without causing any undesirable biological effects or interacting in a deleterious manner with any of the other components of the composition in which it is contained. When the term “pharmaceutically acceptable” is used to refer to a pharmaceutical excipient, it is implied that the carrier or excipient has met the required standards of toxicological and manufacturing testing and/or that it is included on the Inactive Ingredient Guide prepared by the U.S. Food and Drug administration.

“Pharmacologically active” (or simply “active”) as in a “pharmacologically active” analog, refers to a compound having the same type of pharmacological activity as the parent compound and approximately equivalent in degree.

As used herein, the term “patient” or “individual” or “subject” refers to any person or mammalian subject for whom or which therapy is desired, and generally refers to the recipient of the therapy to be practiced according to the invention.

The active agent in the present controlled release formulation is one that is “moisture sensitive,” meaning that the agent undergoes a chemical or physical change in the presence of moisture that undesirably alters its physical, chemical, and/or pharmacological properties. An active agent may undergo such a change as a result of a hydrolytically labile linkage or functionality in its molecular structure, and/or as a result of a moisture-sensitive contaminant associated with an otherwise moisture-stable active agent. Topiramate is believed to be an example of the latter class; see Micheel et al. (1998), supra. Within the former class, representative moisture-sensitive active agents include, without limitation, aspirin, niacinamide, bupropion, ranitidine, nicorandil, triflusal, cilazapril, tiotropium, venlafaxine, trimetazidine, tramadol, metformin, and the like. The present invention provides for a chemically and physically stable orally administrable controlled release formulation of a moisture-sensitive active agent, in which hydrophilic excipients are excluded and contact between the active agent and moisture is both delayed and minimized.

Suitable moisture-sensitive active agents that may be incorporated into the present formulations include, but are not limited to, agents from the following therapeutic classes: analeptic agents; analgesic agents; anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents; antimigraine preparations; antinauseants; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, antianginal agents, central nervous system (CNS) agents, beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; ophthalmic drugs such as antiglaucoma agents; parasympatholytics; peptide drugs; psychostimulants; sedatives; steroids, including progestogens, estrogens, corticosteroids, androgens and anabolic agents; smoking cessation agents; sympathomimetics; tranquilizers; and vasodilators including general coronary, peripheral and cerebral. A preferred active agent is topiramate.

The active agent may be used as synthesized and/or obtained from the manufacturer, or it may be subject to comminution or micronization. The amount of active agent in a unit dosage form prepared from the present formulation will depend, of course, on the particular active agent, indication, patient, and the like; in general, for topiramate, a preferred daily dosage is in the range of about 20 mg to about 150 mg, generally in the range of about 20 mg to about 100 mg, with representative daily dosages thus including, for instance, 20 mg, 23 mg, 25 mg, 30 mg, 40 mg, 46 mg, 50 mg, 55 mg, 60 mg, 65 mg, 69 mg, 75 mg, 80 mg, 85 mg, 90 mg, 92 mg, 95 mg, 97 mg, and 100 mg. A unit dose in the formulation may be equivalent to the daily dose, or it may be half, one third, or one quarter of the daily dose. In a preferred embodiment, the unit dose is equivalent to the daily dose.

The excipients of the present sustained release formulation are all non-hygroscopic in nature, thus substantially preventing absorption of moisture during storage of the compressed dosage forms, such as tablets, prepared from the formulation. The use of non-hygroscopic excipients, without inclusion of any hygroscopic excipients or materials prolongs the shelf life of the dosage forms herein. This is particularly important with moisture-sensitive pharmacologically active agents, as in the present dosage forms, in which the agent is embedded in a non-hygroscopic structure and thus protected from contact with external moisture.

Two primary formulation components interact to provide the sustained release mechanism. The first component is a lipidic matrix-forming excipient having an octanol: water partition coefficient P greater than 1.0, preferably greater than 5.0, and a melting point greater than about 40° C., preferably greater than about 60° C. The lipidic matrix-forming excipient is thus solid but somewhat malleable at ambient temperature, and serves to hold a three-dimensional shape upon tablet formation via direct compression or roller compaction. The second component is a water-soluble, non-hygroscopic channel-forming excipient that begins dissolving upon exposure of the tablet surface to water, gradually forming narrow channels extending from the tablet surface inward, within the body of the aforementioned lipidic matrix, thus enabling sustained release of the active agent via the channels formed.

The lipidic matrix-forming excipient is typically a waxy, malleable lipidic compound or composition that, like all of the excipients incorporated in the formulation, is non-hygroscopic. Ideally, although not necessarily, the lipidic matrix-forming excipient is used in particulate form or in the form of small pellets, with a substantially homogeneous particle or pellet size, to facilitate complete, homogeneous mixing with other excipients prior to compression. Any such lipidic excipient can be used, providing that it is non-hygroscopic as well as compatible and chemically inert with respect to the other components of the formulation.

Suitable lipidic matrix-forming excipients include, by way of example, C₁₂-C₂₆ lipids, preferably saturated C₁₂-C₂₆ lipids such as saturated C₁₂-C₂₆ fatty acids and derivatives and combinations thereof; waxes; and gums. Generally preferred lipidic matrix-forming excipients are saturated C₁₂-C₂₆ lipids, e.g., saturated C₁₂-C₂₆ fatty acids, and derivatives thereof that mask the hydroxyl functionality within the carboxylic acid group, including esters, diesters, ketones, and the like. Preferred such derivatives are glyceryl mono-, di- and tri-esters of C₁₂-C₂₆ fatty acids, preferably esters of saturated C₁₂-C₂₆ fatty acids; and propylene glycol mono- and di-esters of C₁₂-C₂₆ fatty acids, again, preferably esters of saturated C₁₂-C₂₆ fatty acids. Saturated C₁₂-C₂₆ fatty acids include lauric acid (C₁₂: CH₃(CH₂)₁₀COOH), myristic acid (C₁₄: CH₃(CH₂)₁₂COOH), palmitic acid (C₁₆: CH₃(CH₂)₁₄COOH), stearic acid (C₁₈: CH₃(CH₂)₁₆COOH), arachidic acid (C₂₀: CH₃(CH₂)₁₈COOH), behenic acid (C₂₂: CH₃(CH₂)₆COOH), lignoceric acid (C₂₄: CH₃(CH₂)₂₂COOH), and cerotic acid (C₂₆: CH₃(CH₂)₂₄COOH). Glyceryl di-esters and tri-esters may respectively comprise two or three of the same fatty acid chain (e.g., glyceryl distearate), or they may comprise two or three different fatty acid chains (e.g., glyceryl dipalmitostearate). Similarly, propylene glycol di-esters may contain two identical fatty acid chains or two different fatty acid chains. Other esters of fatty acids may also be advantageously employed providing that they exhibit the required physical properties, i.e., existing as malleable solids at ambient temperature, and hydrophobic as defined above. Such esters include diesters formed from two C₁₂-C₂₆ fatty acid chains and a linear diol, e.g., a long-chain hydrophobic diol; and polyglycerized fatty acids. Particularly preferred lipidic matrix-forming excipients for use herein are glyceryl mono-, di-, and tri-esters of saturated C₁₂-C₂₆ fatty acids. Examples of the latter include, without limitation, glyceryl behenate (e.g., Compritol®E ATO or Compritol®888 ATO, from Gattefosse), glyceryl distearate (e.g., Precirol® ATOS), glyceryl palmitostearate, glyceryl dipalmitostearate (Biogapress Vegetal BM297 ATO), and the like. Other suitable lipidic excipients include carnauba wax, beeswax, and petroleum.

The channel-forming excipient is water soluble, i.e., it has a solubility in water in the range of about 5% by weight to greater than about 30% by weight, more preferably in the range of about 10% by weight to greater than about 25% by weight. At the same time, the channel-forming excipient should be non-hygroscopic. Ideal materials for use as the channel-forming excipient are sugar alcohols (glycitols) such as mannitol, sorbitol, galactitol, fucitol, iditol, inositol, and volemitol, as well as disaccharides and higher saccharides formed with other sugars, particularly the glucose-sorbitol disaccharide (alpha-D-glucopyranosido-1,6-sorbitol) and the glucose-mannitol disaccharide (alpha-D-glucopyranosido-1,6-mannitol), as well as combinations thereof. One such particularly preferred composition is the equimolar mixture of alpha-D-glucopyranosido-1,6-sorbitol and alpha-D-glucopyranosido-1,6-mannitol known as “isomalt,” available commercially as galenIQ® from BENEO-Palatinit.

The ratio of channel-forming excipient to lipidic matrix-forming excipient is generally in the range of about 1.5:1 to about 6:1. Within this range, lower ratios on the order of about 1.5:1 to about 4:1 are generally preferred for lower doses of active agent and larger active agent particles, and higher ratios, up to about 6:1, are preferred for higher doses of active agents and smaller, e.g., micronized, active agent particles. Together, the lipidic matrix-forming excipient and the channel-forming excipient represent about 20 wt. % to about 80 wt. %, preferably about 25 wt. % to about 40 wt. %, of the formulation and any dosage form prepared therefrom.

The formulation also includes a non-hygroscopic filler as well as manufacturing aids that serve as lubricants and/or flow enhancers and/or impart elasticity to the tablet, preventing tablet cracking during and after compression and preventing need for a high ejection force.

Suitable non-hygroscopic fillers include anhydrous salts such as anhydrous sodium sulfate, anhydrous calcium hydrogen phosphate, and the like; it should be emphasized, however, that any non-hygroscopic filler may be used. Non-hygroscopic calcium sulfate dihydrate such as that available from JRS Pharma as COMPACTROL® is preferred.

Microcrystalline cellulose is an optimal manufacturing aid, providing it is treated so as to be non-hygroscopic, e.g., by silicification; Prosolv SMCC®90, JRS Pharma is an example of one such silicified microcrystalline cellulose excipient. Other manufacturing aids include magnesium stearate, calcium stearate, sodium stearyl fumarate, micronized silica, and fumed silica (e.g., (AEROSIL R972®, Evonik).

Any suitable process may be employed in the preparation of the aforementioned formulations and dosage forms derived therefrom, provided that the process is dry and that dosage forms are prepared with compressive force. Techniques such as wet granulation and extrusion must obviously be avoided.

Dry processes include, first of all, direct compression in which: (1) an excipient mixture is prepared by sieving and then dry-blending all excipients, with the exception of lubricants and manufacturing aids (e.g., magnesium stearate, silicas); (2) incorporating the active agent into the excipient mixture and continuing to blend for an additional 10 to 15 minutes; (3) incorporating manufacturing aids such as magnesium stearate and silicas and continuing to blend for an additional 10 to 15 minutes; and (4) tableting the final mixture by compressing it on a suitable tablet press.

Roller compaction is an alternative dry formulation process in which steps (1), (2), and (3) above are carried out, and the resulting dry mixture is processed through a conventional roller compactor to form ribbons, which are thereafter screened down to granules.

Other techniques may also be used to prepare solid dosage forms from the present formulations, provided that they are dry techniques, preventing or at least minimizing contact of formulation components with moisture during manufacture.

In another embodiment, a bilayer tablet is provided that is composed of a first layer that provides for controlled release of a moisture-sensitive active agent, and a second layer that provides for immediate release of a second active agent. The first layer is composed of the controlled release formulation described above, while the second layer contains a second active agent that may or may not be moisture-sensitive, and contains the same or substantially the same excipients as employed for the first layer.

The second active agent may be selected from the same classes of drug as specified for the moisture-sensitive active agent. The second active agent may be employed to enhance the pharmacological activity of the first drug, to reduce unwanted side effects resulting from administration of the first drug, to reduce the required dosage of the first drug, to increase efficacy relative to monotherapy with the first drug, or to provide an entirely different therapeutic effect. In one exemplary embodiment, the second drug is an appetite suppressant administered in combination with topiramate as the first drug.

Examples of suitable appetite suppressants that can be employed as the second drug include, for instance, sympathomimetic amines. Sympathomimetic amines, including the catecholamines, are amine drugs that mimic the actions of drugs that activate the sympathetic nervous system, such as epinephrine and norepinephrine. Sympathomimetic amines thus include amphetamine, benzphetamine, bupropion, chlorphentermine, colterol, diethylpropion, dopamine, dobutamine, ephedrine, epinephrine, epinine, ethylnorepinephrine, fenfluramine, fenoldapam, hydroxyamphetamine, ibopamine, isoetharine, isoproterenol, mephentermine, metaproterenol, metaraminol, methoxamine, methoxyphenamine, midodrine, norepinephrine, phendimetrazine, phenmetrazine, phentermine, phenylephrine, phenylethylamine, phenylpropanolamine, prenalterol, propylhexedrine, protokylol, ritodrine, terbutaline, tuaminoheptane, tyramine, and acid addition salts thereof, either organic or inorganic. Common acid addition salts of some of the aforementioned sympathomimetic amines include, without limitation, dobutamine hydrochloride, epinephrine bitartrate, ethylnorepinephrine hydrochloride, fenoldopam mesylate, hydroxyamphetamine hydrobromide, isoproterenol hydrochloride, mephentermine sulfate, metaraminol bitartrate, methoxamine hydrochloride, norepinephrine bitartrate, phenylephrine hydrochloride, and terbutaline sulfate.

Preferably, the sympathomimetic amine is phentermine, chlorphentermine, or bupropion, with phentermine and bupropion particularly preferred. In an exemplary embodiment, the moisture-sensitive active agent is topiramate and the sympathomimetic amine administered is phentermine, wherein the daily dose of topiramate is as given above for the monotherapeutic regimen, and the corresponding daily dose of phentermine that is co-administered is such that the weight ratio of the daily dose of topiramate to the daily dose of phentermine is in the range of about 2.5:1 to about 20:1, typically in the range of about 5:1 to about 20:1. The amount of phentermine or any other second agent in a unit dosage form will depend, again, on the particular active agent, indication, patient, and the like; for phentermine, a preferred daily dosage is in the range of about 2.5 mg to about 20 mg, generally in the range of about 2.5 mg to about 17.5 mg, with representative daily dosages thus including, for instance, 2.5 mg, 3.75 mg., 5.0 mg; 7.5 mg; 10 mg; 11.75 mg; 12.5 mg; 15 mg; and 17.5 mg. A unit dose in the formulation may be equivalent to the daily dose, or it may be half, one third, or one quarter of the daily dose. In a preferred embodiment, the unit dose is equivalent to the daily dose.

In another exemplary embodiment, the moisture-sensitive active agent is topiramate and the second active agent is bupropion, wherein the daily dose of topiramate is as given above for the monotherapeutic regimen, and the corresponding daily dose of bupropion that is co-administered is such that the weight ratio of the daily dose of topiramate to the daily dose of bupropion is in the range of about 1:5 to about 3:1, preferably in the range of about 1:4 to about 2:1, most preferably in the range of about 1:4 to about 1.5:1.

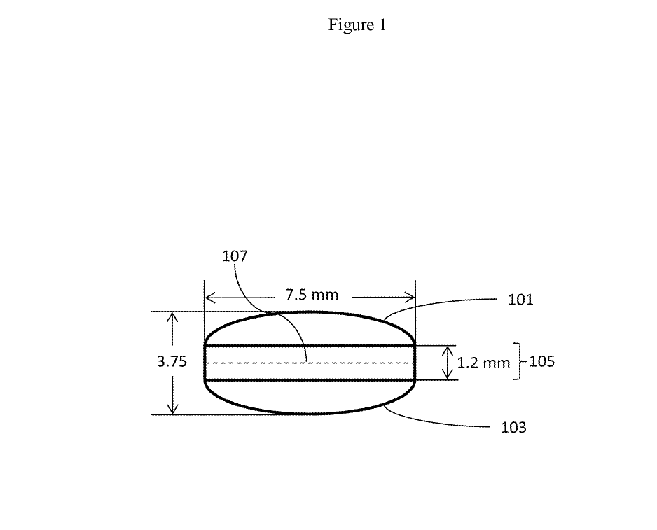

Fabrication of the bilayer tablet: Ideally, the immediate release layer with the second active agent is compressed with the controlled release layer using a compaction force sufficient to enable physical attachment of the two layers. The two layers are preferably arranged so that the area of the interface is maximized relative to the dimensions of the tablet. A side view of an exemplary tablet is shown in FIG. 1. The tablet has an upper portion 101 and a lower portion 103 that are curved and dome shaped. There is a central section 105 representing a cylindrical portion. The dashed line 107 represents the midline bisecting the tablet along the dimension shown. Roughly speaking the midline 107 may be the approximate location of the interface between the two layers of the tablet, but the interface is preferably within the central section 105, this maximizes the surface area of the interface and thereby maximizes binding between the two layers. Exemplary dimensions are shown. A tablet as shown, with a height of 3.75 mm and a width of 7.5 mm and having a 1.2 mm height for the central section 105, provides a geometric factor of 1.145. This size may be optimal for tablets having 46 mg topiramate. The density may be about 1.274. In another example, for a tablet containing 23 mg topiramate, the dimensions may be 2.35 mm in height, 5.6 mm in width and 1.05 mm height for the central section. Those dimensions provide for a geometric factor of about 1.25. The density may be about 1.286.

In a preferred embodiment, the formulations for the two layers are prepared using substantially the same excipients, such that only minor modifications or additions are necessary to finalize each formulation. For instance, the controlled release layer and the immediate release layer can both be formulated using the same non-hygroscopic filler (e.g., non-hygroscopic calcium sulfate dihydrate such as that available from JRS Pharma as COMPACTROL®), the same lubricants and manufacturing aids (e.g., magnesium stearate, fumed silica, and non-hygroscopic, silicified microcrystalline cellulose), and the same water soluble channel-forming excipient (galenIQ® isomalt from BENEO-Palatinit). In this way, a single blend of the aforementioned excipients may be prepared, the blend may be divided into two batches, one intended for the controlled release formulation and the other intended for the immediate release formulation, and the appropriate additions then made: the lipidic matrix-forming excipient is added into the controlled release formulation along with the moisture sensitive active agent; and the second active agent is added into the immediate release formulation. The two formulations can then be finally mixed and compressed or compacted into two layers of a bilayer tablet. See also Moodley et al., Int J Mol Sci. 2012; 13(1); 18-43 for a review of geometric configurations for controlled drug delivery.

### Example 1

A compressed tablet formulation for the controlled release of topiramate was prepared as follows. Calcium sulfate dihydrate (Compactrol®, JRS Pharma), glyceryl behenate (Compritol®E ATO, Gattefosse), silicified microcrystalline cellulose (Prosolv 90®, JRS Pharma), and isomalt (galenIQ® 721, BENEO-Palatinit, sieved through Mesh No. 80), in the quantities set forth in Table 1, below, were dry-blended together. Magnesium stearate was added, followed by hydrophobic colloidal silica (Aerosil R972®, Evonik), with several minutes of mixing after each addition. Finally, micronized topiramate (ScinoPharm, Tainan, Taiwan) was added. Mixing was carried out for about 15 minutes, to ensure homogeneity.

TABLE 1

| COMPONENT | WT, MG | WT. % |
|---|---|---|
| Topiramate | 1000.0 | 20.00% |
| Compactrol | 1130.0 | 22.60% |
| Compritol E ATO | 600.0 | 12.00% |
| Prosolv 90 | 1205.0 | 24.10% |
| galenIQ 721 | 700.0 | 20.00% |
| Mg stearate | 40.0 | 0.80% |
| Aerosil R972 | 25.0 | 0.50% |
| Total | 5000.0 | 100.00% |

Six individual tablets of the foregoing formulation, each containing 23 mg topiramate, were then prepared using direct compression in a tablet press at a compaction force of 2.5 tons (25 kn). The average weight of each tablet was 114.2 mg, with an average hardness of 7.02 kP. Dissolution test results are provided in Table 7 and FIG. 1.

### Example 2

A compressed tablet formulation for the controlled release of topiramate was prepared with the same components as in Example 1. The quantities of the excipients and active agent set forth in Table 2:

TABLE 2

| COMPONENT | WT, MG | WT. % |
|---|---|---|
| Topiramate | 1000.0 | 20.00% |
| Compactrol | 1130.0 | 22.60% |
| Compritol | 700.0 | 14.00% |
| Prosolv 90 | 1205.0 | 24.10% |
| galenIQ | 900.0 | 18.00% |
| Mg stearate | 40.0 | 0.80% |
| Aerosil R972 | 25.0 | 0.50% |
| Total | 5000.0 | 100.00% |

Six individual tablets of the foregoing formulation, each containing 23 mg topiramate, were then prepared using direct compression in a tablet press at a compaction force of 2.5 tons (25 kn). The average weight of each tablet was 115.3 mg, with an average hardness of 6.5 kP. Dissolution test results are provided in Table 7 and FIG. 1.

### Example 3

A compressed tablet formulation for the controlled release of topiramate was prepared with the same components as in Example 1. The quantities of the excipients and active agent set forth in Table 3:

TABLE 3

| COMPONENT | WT, MG | WT. % |
|---|---|---|
| Topiramate | 1000.0 | 20.00% |
| Compactrol | 1130.0 | 22.60% |
| Compritol | 900.0 | 18.00% |
| Prosolv 90 | 1205.0 | 24.10% |
| galenIQ | 700.0 | 14.00% |
| Mg stearate | 40.0 | 0.80% |
| Aerosil R972 | 25.0 | 0.50% |
| Total | 5000.0 | 100.00% |

Six individual tablets of the foregoing formulation, each containing 23 mg topiramate, were then prepared using direct compression in a tablet press at a compaction force of 2.5 tons (25 kn). The average weight of each tablet was 115.2 mg, with an average hardness of 5.06 kP. Dissolution test results are provided in Table 7 and FIG. 1.

### Example 4

A compressed tablet formulation for the controlled release of topiramate was prepared with the same components as in Example 1. The quantities of the excipients and active agent set forth in Table 4:

TABLE 4

| COMPONENT | WT, MG | WT. % |
|---|---|---|
| Topiramate | 1794.0 | 35.88% |
| Compactrol | 661.0 | 13.22% |
| Compritol | 325.0 | 6.50% |
| Prosolv 90 | 705.0 | 14.10% |
| galenIQ | 1450.0 | 29.00% |
| Mg stearate | 40.0 | 0.80% |
| Aerosil R972 | 25.0 | 0.50% |
| Total | 5000.0 | 100.00% |

Six individual tablets of the foregoing formulation, each containing 92 mg topiramate, were then prepared using direct compression in a tablet press at a compaction force of 2.5 tons (25 kn). The average weight of each tablet was 256.2 mg, with an average hardness of 9.00 kP. Dissolution test results are provided in Table 7 and FIG. 1.

### Example 5

A compressed tablet formulation for the controlled release of topiramate was prepared with the same components as in Example 1. The quantities of the excipients and active agent set forth in Table 5:

TABLE 5

| COMPONENT | WT, MG | WT. % |
|---|---|---|
| Topiramate | 1794.0 | 35.88% |
| Compactrol | 661.0 | 13.22% |
| Compritol | 425.0 | 8.50% |
| Prosolv 90 | 705.0 | 14.10% |
| galenIQ | 1350.0 | 27.00% |
| Mg stearate | 40.0 | 0.80% |
| Aerosil R972 | 25.0 | 0.50% |
| Total | 5000.0 | 100.00% |

Six individual tablets of the foregoing formulation, each containing 92 mg topiramate, were then prepared using direct compression in a tablet press at a compaction force of 2.5 tons (25 kn). The average weight of each tablet was 255.4 mg, with an average hardness of 8.50 kP. Dissolution test results are provided in Table 7 and FIG. 1.

### Example 6

A compressed tablet formulation for the controlled release of topiramate was prepared with the same components as in Example 1. The quantities of the excipients and active agent set forth in Table 6:

TABLE 6

| COMPONENT | WT, MG | WT. % |
|---|---|---|
| Topiramate | 1794.0 | 35.88% |
| Compactrol | 661.0 | 13.22% |
| Compritol | 625.0 | 12.50% |
| Prosolv 90 | 705.0 | 14.10% |
| galenIQ | 1150.0 | 23.00% |
| Mg stearate | 40.0 | 0.80% |
| Aerosil R972 | 25.0 | 0.50% |
| Total | 5000.0 | 100.00% |

Six individual tablets of the foregoing formulation, each containing 92 mg topiramate, were then prepared using direct compression in a tablet press at a compaction force of 2.5 tons (25 kn). The average weight of each tablet was 255.2 mg, with an average hardness of 7.35 kP.

### Example 7

A compressed tablet formulation for the controlled release of topiramate was prepared with the same components as in Example 1. The quantities of the excipients and active agent set forth in Table 7:

TABLE 7

| COMPONENT | WT, MG | WT. % |
|---|---|---|
| Topiramate | 1923.0 | 38.46% |
| Compactrol | 1260.5 | 25.21% |
| Compritol | 305.0 | 6.10% |
| Prosolv 90 | 750.0 | 15.00% |
| galenIQ | 1446.50 | 28.093% |
| Mg stearate | 40.0 | 0.80% |
| Aerosil R972 | 25.0 | 0.50% |
| Total | 5000.0 | 100.00% |

Six individual tablets of the foregoing formulation, each containing 92 mg topiramate, were then prepared using direct compression in a tablet press at a compaction force of 2.5 tons (25 kn). The average hardness of the tablets was 14.67 kP.

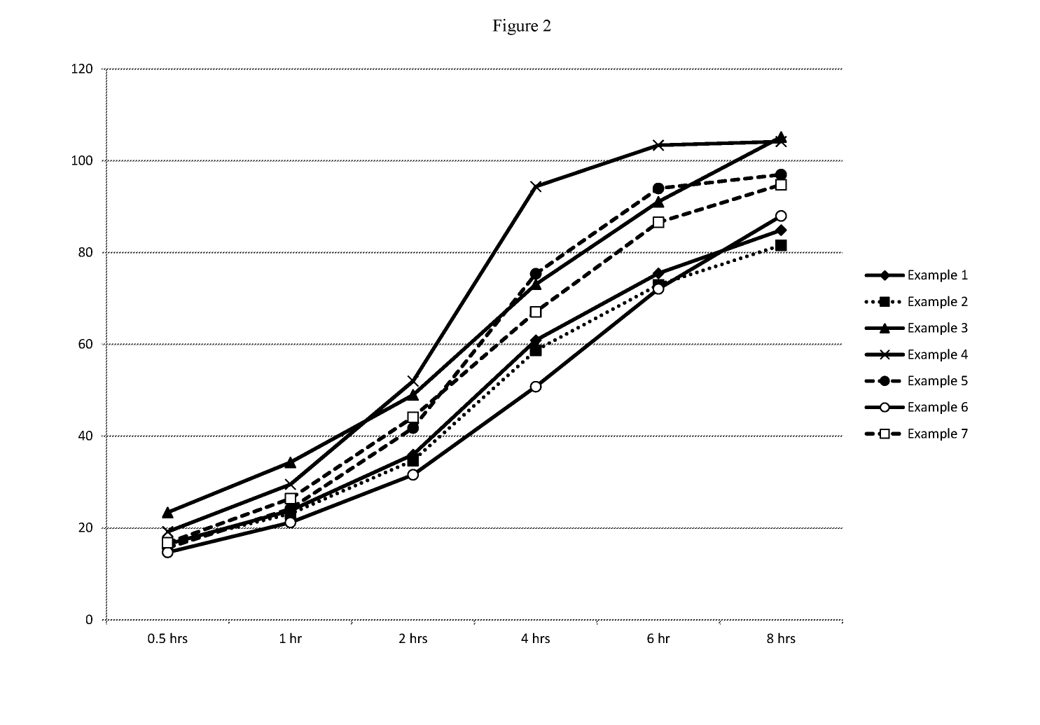

Table 8 provides percent dissolution over time for the tablets described in Examples 1-7. The data is shown graphically in FIG. 2.

TABLE 8

| | | Example 1 | Example 2 | Example 3 | Example 4 | Example 5 | Example 6 | Example 7 |
|---|---|---|---|---|---|---|---|---|
| 0.5 | hrs | 16.5 | 16.6 | 23.4 | 19.2 | 15.7 | 14.7 | 16.8 |
| 1 | hr | 23.8 | 23.1 | 34.3 | 29.5 | 24.2 | 21.2 | 26.4 |
| 2 | hrs | 36 | 34.7 | 49 | 52 | 41.8 | 31.6 | 44.16 |
| 4 | hrs | 60.9 | 58.7 | 73.1 | 94.4 | 75.4 | 50.8 | 67.13 |
| 6 | hrs | 75.5 | 73 | 91.1 | 103.4 | 94 | 72.1 | 86.6 |
| 8 | hrs | 84.9 | 81.6 | 105.2 | 104.2 | 97 | 88 | 94.8 |

We claim:

1. An orally administrable compressed tablet for controlled release of a moisture-sensitive pharmacologically active agent, comprising:
a non-hygroscopic, lipidic matrix-forming excipient having a melting point greater than about 40° C.;
a non-hygroscopic, water-soluble, channel-forming excipient;
a non-hygroscopic filler; and
a therapeutically effective amount of the pharmacologically active agent, wherein the tablet is substantially free of hygroscopic excipients.

2. The compressed tablet of claim 1, wherein the lipidic matrix-forming excipient has a melting point greater than about 60° C.

3. The compressed tablet of claim 1, wherein the lipidic matrix-forming excipient is selected from C₁₂-C₂₆ fatty acids, C₁₂-C₂₆ fatty acid esters, waxes, gums, and combinations thereof.

4. The compressed tablet of claim 3, wherein the lipidic matrix-forming excipient is selected from saturated C₁₂-C₂₆ fatty acids, glyceryl mono-, di- and tri-esters of saturated C₁₂-C₂₆ fatty acids, propylene glycol mono- and di-esters of saturated C₁₂-C₂₆ fatty acids, waxes, gums, and combinations thereof.

5. The compressed tablet of claim 4, wherein the lipidic matrix-forming excipient is selected from glyceryl mono, di- and tri-esters of C₁₂-C₂₆ fatty acids and combinations thereof.

6. The compressed tablet of claim 5, wherein the lipidic matrix-forming excipient is selected from glyceryl behenate, glyceryl distearate, glyceryl palmitostearate, glyceryl dipalmitostearate, and combinations thereof.

7. The compressed tablet of claim 1, wherein the channel-forming excipient is selected from sugar alcohols, sugar alcohol disaccharides, disaccharides of a sugar alcohol and a monosaccharide, and combinations thereof.

8. The compressed tablet of claim 7, wherein the channel-forming excipient is selected from mannitol, sorbitol, galactitol, fucitol, iditol, inositol, volemitol, alpha-D-glucopyranosido-1,6-sorbitol, alpha-D-glucopyranosido-1,6-mannitol, isomalt, and combinations thereof.

9. The compressed tablet of claim 1, wherein the non-hygroscopic filler is selected from anhydrous inorganic salts and calcium sulfate dihydrate.

10. The compressed tablet of claim 1, further comprising at least one additional non-hygroscopic excipient that comprises a manufacturing aid selected from lubricants, flow enhancers, and compression aids.

11. The compressed tablet of claim 1, wherein a ratio of the channel-forming excipient to the lipidic matrix-forming excipient is in the range of about 1.5:1 to about 6:1, and together represent in the range of about 20 wt. % to about 80 wt. % of the tablet.

12. The compressed tablet of claim 11, wherein the channel-forming excipient and the lipidic matrix-forming excipient together represent in the range of about 25 wt. % to about 40 wt. % of the tablet.

13. The compressed tablet of claim 1, wherein:
the pharmacologically active agent is topiramate;
the non-hygroscopic, lipidic matrix-forming excipient having a melting point greater than about 40° C., is selected from glyceryl mono-esters, di-esters, and tri-esters of C₁₂-C₂₆ fatty acids; and,
the non-hygroscopic, water-soluble, channel-forming excipient is selected from mannitol, sorbitol, galactitol, fucitol, iditol, inositol, volemitol, alpha-D-glucopyranosido-1,6-sorbitol, alpha-D-glucopyranosido-1,6-mannitol, isomalt, and combinations thereof.

14. An orally administrable bilayer tablet for controlled release of topiramate and immediate release of phentermine, comprising:
(a) a first layer comprising
a controlled release layer of a non-hygroscopic, lipidic matrix-forming excipient having a melting point greater than about 40° C.;
a non-hygroscopic, water-soluble, channel-forming excipient;
a non-hygroscopic filler; and
a therapeutically effective amount of topiramate, wherein the first layer is substantially free of hygroscopic excipients; and,
(b) adhered to the first layer, a second layer comprising:
a non-hygroscopic, water-soluble, channel-forming excipient;
a non-hygroscopic filler; and
a therapeutically effective amount of phentermine, wherein the second layer is substantially free of hygroscopic excipients.

15. The orally administrable bilayer tablet of claim 14, wherein the therapeutically effective amount of topiramate is between 20 mg and 100 mg of topiramate and wherein the therapeutically effective amount of phentermine is between 3 mg and 20 mg of phentermine, or between 3.73 mg and 24.88 mg of phentermine hydrochloride.

16. A method for treating a patient for a condition selected from obesity, overweight, diabetes, sleep apnea, and coronary artery disease, comprising orally administering to the patient the bilayer tablet of claim 14.

17. A method for treating a patient for a condition selected from obesity, overweight, diabetes, sleep apnea, and coronary artery disease, comprising orally administering to the patient the bilayer tablet of claim 15.

18. A process for manufacturing an orally administrable compressed tablet for controlled release of a moisture-sensitive pharmacologically active agent and immediate release of a second pharmacologically active agent, comprising:
blending, in the absence of moisture and in particulate form, a non-hygroscopic, water-soluble, channel-forming excipient, a non-hygroscopic filler, and manufacturing aids, to form an initial excipient mixture;
dividing the initial excipient mixture into a first batch and a second batch;
incorporating a lipidic matrix-forming excipient and a moisture-sensitive active agent into the initial excipient mixture, to form a controlled release formulation;
incorporating a second active agent into the second batch, to form an immediate release formulation;
applying compressive force to a contained quantity of the controlled release formulation to form a controlled release layer;
applying compressive force to a contained quantity of the immediate release formulation to form an immediate release layer; and
compressing the immediate release layer to the controlled release layer in a manner that facilitates bonding of the two layers.

19. The process of claim 18, wherein the moisture-sensitive pharmacologically active agent is topiramate and the second pharmacologically active agent is phentermine.

20. The process of claim 19, wherein the amount of topiramate in the tablet is between 20 mg and 100 mg and wherein the amount of phentermine in the tablet is between about 3 mg and 20 mg of phentermine, or between about 3.7 mg and 24.9 mg of phentermine hydrochloride.
